(19) United States
(12) Patent Application Publication
(10) Pub. No.: US 2013/0280350 A1
(43) Pub. Date: Oct. 24, 2013

(54) HERBAL TOPICAL FORMULATION

(71) Applicants: Jayashree Rajan, Chennai (IN); Balakrishnan Rajan, Chennai (CN)

(72) Inventors: Jayashree Rajan, Chennai (IN); Balakrishnan Rajan, Chennai (CN)

(73) Assignee: Sequent Scientific Limited, Bangalore (IN)

(21) Appl. No.: 13/881,864

(22) PCT Filed: Jun. 8, 2010
(86) PCT No.: PCT/IN10/00378

§ 371 Date: Apr. 26, 2013

(51) Int. Cl. A61K 47/46

(52) U.S. Cl. CPC A61K 47/46
USPC 424/725; 514/783

(57) ABSTRACT

A topical herbal composition comprising aqueous extract of leaf of Datura species alongwith pharmaceutically acceptable vehicle useful for the eradication of Lice (and nits) in human and Ticks (eggs and larvae) in animals wherein the said herbal extract is detoxified with neem is disclosed herein.

### TECHNICAL FIELD OF INVENTION

The present invention relates to herbal insecticide/pediculocide/acaricides for topical administration. More particularly, the invention relates to an herbal formulation comprising an herbal extract from the species Datura species, and more specifically Datura Alba, with pharmaceutically acceptable vehicle, useful for eradication of Lice (and nits) in human and Ticks (eggs and larvae), in animals. The invention further relates to the process of detoxification of a toxic herb comprising using Neem or Neem based composition for detoxifying prior to formulation and administration.

### BACKGROUND AND PRIOR ART OF INVENTION

Ticks are among the most important vectors of human and animal pathogens including arboviruses, rickettsiae, spirochetes, parasitic protozoa and possibly nematodes. (Sonenshine, D.E. (1993). Biology of Ticks, Volume 2 (Oxford University Press: Oxford)). The incidence of tick borne disease has risen in recent years and is considered to be a major public health problem. Some species of tick secrete a paralytic toxin capable of disabling or killing their host. Furthermore, severe infestations can result in host such as anemia, loss of appetite, weakening of the immune system, disruption of liver metabolism and excessive hair loss (Nelson, W. A. et. al. (1977).

Ticks are divided into three families: Nuttalliellidae, Ixodidae and Argasidae. Among the three, ticks of the family Ixodidae are the most damaging to humans and animals alike. Representative of the Ixodids include the livestock ravaging cattle ticks, Boophlius microplus and Amblyomma hebraeum, the lyme disease transmitting deer tick, Ixodes scapulans, and the typhus and tularaemia transmitting lone star tick, Amblyomma americanum.

Ticks can detect heat emitted or carbon dioxide respired from a nearby host and also the changes in temperature and day length are some of the factors signaling a tick to seek a host. Ticks have a harpoon-like structure in their mouth area, known as a hypostome that allows them to anchor themselves firmly in place while feeding. The hypostome has a series of barbs angled back, which is why they are so difficult to remove once they have penetrated a host. Ticks lay eggs away from their hosts. 10,000 to 15,000 eggs are laid and more than 80% of them survive and find their way back to hosts for feeding.

One way to prevent Tick infestation is to control the tick population by use of chemicals called acaricides. However, chemical control using acaricides poses significant problems for the environment and public health. In addition, ticks are rapidly developing resistance to the chemicals used, making this approach of poor efficacy in the long term as well as labour intensive.

Further, Head louse infestations are prevalent worldwide. About 16 to 17% of people in India have been infested with head lice. Similarly, in a large number of developing and under developed countries, ticks, fleas and lice pose serious health problems.

In general, head lice or obligate parasites spend their entire life on the host scalp and feed exclusively on blood 4 to 5 times daily. Any part of the scalp may be colonized by lice, however, lice favour the nap of the neck and area behind the ears where they mostly lay their eggs.

Lice are arthropods belonging to the order Phthiraptera (wingless insects), specifically to the suborder Anoplura (sucking lice) and to the Pediculidae and Phthiriidae families. Pediculus humanus and Phthirus pubis (pubic lice) are the two species that infest human beings. P. humanus further divides into two subspecies: Pediculus humanus capitis (head louse) and Pediculus humanus corporis (body louse). Infestation with any of these parasites is called pediculosis.

Infestation is spread among people by direct contact or by sharing personal articles such as combs and brushes. Head-head contact is most common mode of lice transmission. During the development of the disease there will be an incubation period, during which the person has no symptoms but is able to spread the disease.

Lice are host specific. Before dying, adult female lice lay from 50 to 100 eggs (nits) on the hair close to the scalp. These eggs hatch into naiads after 4-15 days period through incomplete metamorphosis and the complete cycle takes approximately one month.

It is known that lice have troubled human beings since ancient times and have been found in all cities worldwide. Lice infestation is a serious problem, not only because it causes skin irritation, but also because it transmits diseases such as epidemic typhus, trench fever and so forth.

Pediculosis capitis treatment requires the thorough removal of eggs and lice. To date the main treatment is based on Pyrethrines and Lindane. Permethrin, a synthetic derivate from pyrethrines, is considered the recommended therapy. In general, it is commercially available in the form of a hair conditioner or shampoo. Some of the commercially available permethrins are ovicides and have 7-10 days residual activity. Permethrines must be carefully used in allergic persons, since they may cause breathing disorders.

Treatments with lindane-based products must be carefully applied. The frequent use, repeated applications or ingestion of this substance may cause neurotoxic effects.

There are two systemic agents for treating pediculosis capitis. Trimethoprim-Sulfamethoxazole (80 mg trimethoprim/400 mg sulfamethoxazole) and 200 mcg/kg in a single dose. These systemic agents may be used when there is little or no response to other treatment, and when problems arise in applying topical treatment.

In brief, it may be said that today pediculosis treatment resorts to organochlorine products, organophosphorated products and permethrins, all of them having a high degree of toxicity which prevents the use thereof in pregnant women and calls for special care when applied on children. Moreover, it has been demonstrated that these agents generate a high degree of resistance in lice, thus reducing the effectiveness of treatment. This increased resistance further requires repetition of treatment in shorter time periods.

Therefore, there is unmet need in the art for effective composition for the treatment of pediculosis, which forbids the development of lice resistance yet non toxic to the user.

Therefore, the object of the current invention is to develop a herbal topical composition useful for treating pediculosis in mammal as well as acaricides to prevent ticks in animals.

### SUMMARY OF THE INVENTION

The present invention describes Topical composition based on Herbal extracts of Datura species, which are pretreated for detoxification. In accordance with the above, the present invention provides herbal composition comprising aqueous leaf extract of Datura alba which can be applied topically for the eradication of Lice (and nits) in human and Ticks (eggs and larvae) in animals. Further, the inventive composition is also effective in controlling the dandruff.

The invention further discloses a neem based composition as detoxifying agents (detoxificant) useful for detoxifying toxins that are associated with toxic herbs, used as pharmaceutical, neutraceutical, food and health, wherein the said herbs with high toxicity are pre-treated with neem.

The invention also discloses a process of detoxification of toxic herb like Datura which comprises a step of treating the toxic herb with Neem or Neem based composition for detoxifying prior to formulation and administration. The toxic material of the herbs comprises of leaves, stem, root, flower or any other aerial part of the herb. The detoxified herbal extract further formulated into suitable dosage forms. One such -toxic herb according to the invention is Datura Alba. The Datura Alba Extract is pretreated with a Neem composition for detoxification.

### DETAILED DESCRIPTION OF THE INVENTION

The invention will now be described in detail in connection with certain preferred and optional embodiments, so that various aspects thereof may be more fully understood and appreciated.

Accordingly, the present invention describes a composition for topical application for the eradication of Lice (and nits) in human and Ticks (eggs and larvae) in animals using the leaf extract of Datura alba.The composition is applied topically to the scalp and body.

Datura is a genus of 12-15 species of vespertine flowering plants belonging to the family Solanaceae. Their exact natural distribution is uncertain, due to the extensive cultivation and naturalization throughout the temperate and tropical regions of the globe, however, it is most likely restricted to the Americas, from the United States south through Mexico (where the highest species diversity occurs) to the mid-latitudes of South America. Some species are reported by some authorities to be native to China, but this is not accepted by the Flora of China, where the three species present are treated as introductions from the Americas. It also grows naturally throughout India and most of Australia. According to the old ayurvedic medicinal system (at least since 2000 BC) in India, this plant has versatile uses in medicinal preparations.

The Sanskrit name ‘Datura’ includes all the species and varieties of the plant, but sometimes specifies whether the black or white variety is to be used. Normally, Datura grows in waste lands, along the roadside and railway lines, and in scrub-jungles throughout the tropical parts of India.

Morphology:

It is a spreading herb which sometimes becoming shrubby. Leaves are ovate-lanceolate or broadly ovate, acute or acuminate, unequal at the base and often cordate, entire or dentate, sub-glabrous or with greyish tomentum, generally glandular in a long petiole of up to 10 cm in length. Flowers are often double or triple. Calyx is inflated towards the middle, persistent and reflexes in fruit. Corolla is about twice as long as the calyx, white or tinged with green, pubescent outside and with the 10 toothed limbs. Capsule is globose, tuberculate or muricate, borne on a short thick peduncle, which, unlike that of D. stramonium, is never erect but nodding. It dehisces irregularly exposing a mass of closely packed, light brown, flat seeds which nearly fill the interior.

The datura species available through out the world are as listed below:

Datura affinis ; Datura alba; Datura albidoflava; Datura arborea; Datura aurea; Datura bernhardii; Datura bojeri; Datura capensis; Datura carthaginensis; Datura chlorantha; Datura cornigera; Datura cornucopia; Datura cubensis; Datura ceratocaula; Datura discolor—Desert Thorn-apple; Datura dolichocarpa; Datura dubia; Datura ferox—Long Spined Thorn-apple; Datura fruticosa; Datura gardneri; Datura gigantea ; Datura guayaquilensis; Datura humilis; Datura inermis; Datura inoxia or Datura innoxia—Thorn-apple, downy thorn-apple, Indian-apple, moonflower, sacred datura, toloatzin, or toloache; Datura insignis; Datura kymatocarpa; Datura lanosa; Datura leichhardtii (syn. D. pruinosa)—Leichhardt's Datura; Datura metel; Datura quercifolia;—Oak-leaf Thorn-apple; Datura reburra; Datura suaveolens—Known in Costa Rica as “Reina de la noche” (Night's Queen); Datura stramonium (syn. D. inermis)—Jimsonweed, Thorn-apple; and Datura wrightii—Sacred datura, Sacred Thorn-apple.

The present invention encompasses all the above species. One preferred species according to the invention is Datura alba.

In a preferred embodiment the present invention provides a topical formulation comprising herbal extract of the leaves of Datura Alba. The active therapeutic agent of the present invention is the extract from the leaves of the Datura Alba plant. Latex is extracted in aqueous medium containing active agents which are effective for eradicating Ticks and Lice (including nits, eggs and larvae).

In another preferred embodiment, the present invention describes process for preparation of the extract of datura alba.

The process for preparation of the active extract of Datura alba comprising the steps of:
a) collecting the fresh leaves of Datura Alba and washing with clean pure water followed by immersing the leaves along with Neem leaves in a container containing clean pure water;
b) closing and keeping the container for a minimum period of 6 hours or over night to detoxify datura alba;
c) taking out and cleaning the Datura alba leaves with demineralized water; and
d) putting the wet dripping leaves through the leaf extractor machine to obtain the aqueous extract.

According to this process, 1 kg of Datura alba leaves yield about 250 to 300 ml of extract.

Thus, according to another preferred embodiment, the invention provides a process of detoxification of Datura alba, wherein said process comprises immersing the leaves of Datura alba along with equal quantity of Neem leaves in clean water for a period of 6 to 12 hrs.

In a preferred embodiment, the invention provides a process for preparation of herbal topical composition. Accordingly, the extract is then poured into sterile preferably metallic container, followed by the addition of pharmaceutically acceptable vehicle. The pharmaceutically acceptable vehicle can be selected from natural and synthetic origin which can serve the purpose of effective delivery of the medicament to the target site yet providing the synergy to the composition. The suitable natural vehicles such as like castor oil, linseed oil, mustard oil, cod liver oil, olive oil, coconut oil, sesame oil, or synthetic vehicles such as waxes, glycerine etc, which can be used in an amount of 30 to 80% w/w of the total formulation. The preferred oil according to the invention is coconut oil because of its acceptable aroma, as well as improved efficacy as a vehicle for topical application.

Accordingly, 1 st quality coconut oil is added in about 1 litre to the 300 ml of aqueous extract and boiled the mixture for 2 to 5 hrs, preferably 2 to 3 hrs till the boiled mixture (of 1.3 litres) reduces to about 750 ml or the oil has blended well with the Datura Alba aqua extract.

The formulation is then filtered twice through a specific cloth filter while it is still boiling hot and then left to cool. The cooled mixture is again filtered thrice through the same specified cloth filter and tested for its efficacy on pediculosis.

A typical herbal composition according to the present invention essentially comprises 20 to 40% of the active extract of the leaves of Datura alba together with 30 to 80% of the pharmaceutically acceptable vehicle. The compositions of the present invention essentially comprise gel based or lotion based dosage forms. The selection of pharmaceutical vehicle(s) depends on the desired dosage form.

The herbal extract of the invention is subjected to its toxicity test and found that the herbal extract is absolutely safe as no toxicity is observed. After confirming the toxicity, extensive clinical trials were conducted on human beings and animals with the inventive herbal compositions. It has been proven that the nits, eggs and larvae too were destroyed along with the Lice and Ticks. And there were no adverse side effects noted. Further, the inventive composition is also effective in controlling the dandruff.

Thus the topical herbal formulation is non toxic to human beings as well as to animals.

Another preferred embodiment of the present invention includes the method of treating the subject who is suffering with pediculosis, which method comprises applying topically therapeutically effective amount of the herbal composition. Thus, method for treating pediculosis includes applying topically to the affected skin(body) and scalp area, an effective amount of herbal medicament of the plant extract of the invention. As used herein the term ‘therapeutically effective amount’ means an amount which gives the desired therapeutic effect.

The following examples, which include preferred embodiments, will serve to illustrate the practice of this invention, it being understood that the particulars shown are by way of example and for purpose of illustrative discussion of preferred embodiments of the invention and are in no way limiting the scope of the invention.

### EXAMPLES

### Example 1

Method of Preparation of Formulation:

One kg of Fresh leaves of the Datura Alba were collected and washed with clean pure water. It was then immersed in a container of about 10 litres of clean water and about 1 kg of neem leaves. The container was closed and kept for a minimum period of 6 hours or overnight. The Datura Alba leaves were then taken out and cleaned with Demineralized water and the wet dripping leaves were put through the leaf extractor machine. I kg of Datura Alba leaves yield about 250 to 300 ml of extract.

### Example 2

### Preparation of Herbal Composition:

The extract thus obtained in example 1 was then poured into sterile brass container. One litre of 1st quality coconut oil (pure) was then poured into the same container and boiled for about 2 to 3 hours till the boiled mixture (of 1.3 litres) reduces to about 750 ml or the oil has blended well with the Datura Alba aqua extract.

The formulation was then filtered twice through a specific cloth filter while it is still boiling hot and then left to cool. The cooled mixture was again filtered thrice through the same specified cloth filter.

### Example 3

A herbal formulation comprising following composition was prepared.

| Ingredients | Quantity |
|---|---|
| Aq. Extract of Datura alba | 40% |
| Coconut oil | 60% |

### Example 4

The extensive clinical trials (both Invivo and Invitro) have been carried out with the Govt Vet College and Hospital, Vepery , Chennai. They have done animal studies and used the formulation on dogs and certified that the product is Non Toxic to animals even on ingestion and has stated that not a single adverse side effect was noticed. The animal Welfare Board has certain specifications for animal products and the inventive composition has passed the specification and thus found to be safe.

### Example 5

An Open Clinical Study with the Inventive Product (Licenay-D)

The objective of this study is to examine the pediculicidal efficacy and safety of a natural remedy Licenay-D and to compare it in an open clinical study with a known marketed (control) product called Perlice.

Subjects:
An open clinical study was conducted in Chennai with the invented product

Licenay-D predominantly on children and adults belonging to different classes of society. The lower economical group of children was selected from the Anganwadi of a local elementary school. The children aged between 1-11 years and their parents aged between 21 to 36 years as well were chosen for study.
The children from better socio-economic backgrounds were selected from Sankunni's Ayurvedic clinic & Research Foundation, in different age groups according to the degree of infestations and their willingness.

| | |
|---|---|
| STUDY DESIGN | Open |
| STUDY STATUS | Complete |
| NO OF SUBJECTS FOR | A total of 442 patients took part in this |
| OUR PRODUCT STUDY | clinical |
| | Adults 322: M/F = 38/284 |
| | Children 120: M/F = 40/80 |
| COMPARATIVE STUDY | 10 for Licenay - D, 10 for Perlice |
| DIAGNOSIS DATE | 08.07.2008 to 22.07.2008 |
| DURATION OF STUDY | 45 Days |

Selection of Patients:

Physician examined the infested patients for any dermatological abnormalities and other eye diseases. Healthy children who had not been treated with an anti-lice product during the preceding one month were only included in the study.
Exclusion of a child from the study was: severe scalp dermatosis, secondary infections, and open wounds, tinea of the scalp, eye irritation, conjunctivitis and severe eye diseases.

Diagnosis of Patients:
A team of doctors examined the entire scalp thoroughly with a louse comb and the teeth of the comb should be examined for the presence of living lice after each combing.
In cases, when the child does not allow for combing with a louse comb, the scalp was examined by hand for 5-7 minutes.
The teachers of infested children were given Licenay-D along with clear instructions for its use. The parents of infested children were requested to come to school and were given a consent form (to be filled on a daily basis) along with pediculicide together with instructions for its use.

Method of Application:
The required quantity (about 15-20 ) of the natural remedy Licenay-D which contains coconut oil as a base was applied to the hair of children. After retaining the medication for about an hour, the hair was washed with a mild shampoo and allowed to dry. This procedure was done for a maximum of 3 times in intervals of 3 days.

Examination for Lice:
After drying, the hair was combed with a louse comb for about 3-5 minutes or until the first louse was isolated. In cases where the Child's hair did not allow the use of a louse comb, the scalp was examined by hand for 5-7 minutes. The results were noted carefully in the consent form given prior to its use.
Children and adults were examined a second time one to two days after the first application to determine whether the pediculicide had eradicated all the active stages of the lice.

Content of Licenay-D:
Licenay-D is oil containing natural products-leaves of Datura Alba and coconut oil. This formulation is able to eradicate the lice, nits and dandruff after 2-3 times of applications.

Content of Perlice:
The control product (Allopathic)-Perlice has been sold in India since 1992 and is currently the market leader in Anti Lice. This formulation contains Permethrin, which is a pyrethroid of synthetic origin.

Comparitive Clinical Study of Licenay-D with Perlice
A trial batch of 20 children (girls) with almost equal levels of lice infestation from Anganwadi No 402 was selected for comparative clinical studies with Perlice and Licenay-D. 10 volunteers were randomly selected and treated with Perlice and the other 10 with Licenay-D.

Method of Application:

As mentioned above, the same procedure was followed in both cases and results are as follows:

Licenay-D
6 children showed near total absence of lice and nits after the first application.
2 children showed total absence of lice and nits after 2^{nd} application
1 child stopped usage after 1^{st} application as she did not like the oil on her head.
1 child stopped after 1^{st} application as she developed cold and running nose due to the cooling effect of the product.(Her mother had applied the oil overnight)

Perlice
7 children showed near absence of any lice or nits after 1^{st} application.
2 children stopped as they developed irritation of eyes while applying.
1 child discontinued as she developed acute dryness of scalp which resulted in severe itching. She was given Licenay—D to counter it and is now fine.

### CONCLUSION

After 2-3 applications (for patients with high and severe level of infestation) patients were requested to stop the medication for a month. After 30 days, on examination, it was found that there was no reinfestation. Thus, the herbal based topical composition as described in the invention, was effective in controlling louse infestations under clinical conditions and caused no side effects. The herbal formulation based on extract of Datura, more specifically Aqueous extract of Datura Alba (or other species such as Argomene Mexicana) using a vegetable oil, such as Coconut oil as a vehicle, wherein the Datura Extract is pretreated with Neem leaves in water for detoxification, has been found to be nearly 100% effective in treatment of lice and the like in human beings and Ticks and Mites in warm blooded animals and pets. The said herbal based composition in powder form was also found to be effective not only in treatment of lice and ticks but also for treatment of bugs and mites such as bedbugs by applying on the habitated area such as beds, chairs, sofas in houses as well as public transports and public places.

1. A topical herbal composition comprising aqueous extract of leaf of Datura species alongwith pharmaceutically acceptable vehicle useful for the eradication of Lice (and nits) in human and Ticks (eggs and larvae) in animals wherein the said herbal extract is detoxified with neem.

2. The topical composition as claimed in claim 1, wherein pharmaceutically acceptable vehicle can be selected from natural and synthetic origin.

3. The topical composition as claimed in claim 1, wherein the said herbal extract of leaf of Datura alba is present in an amount of 20 to 40%.

4. The topical composition as claimed in claim 1, wherein natural vehicles are selected from the group of coconut oil, castor oil, linseed oil, mustard oil, cod liver oil, olive oil, sesame oil and synthetic vehicles are selected from the group of waxes, glycerine.

5. The topical composition as claimed in claim 4, wherein the said pharmaceutically acceptable vehicle is present in an amount of 30 to 80%.

6. The topical composition as claimed in claim 1, wherein the said composition is in the form of gel or lotion.

7. The topical composition as claimed in claim 1, wherein the said composition is applied topically to the scalp and body of the subject.

8. A topical composition according to claim 1, wherein said Datura species is Datum alba.

9. A topical herbal composition as claimed in claim 1 comprising herbal extract of leaf of Datura alba is present in amount of 20 to 40% and pharmaceutically acceptable vehicle in an amount of 30 to 80%.

10. The process for preparation of the an active extract of Datura alba comprising the steps of:
a. collecting the fresh leaves of Datura Alba and washing with clean pure water followed by immersing the leaves along with Neem leaves in a container containing clean pure water;
b. closing and keeping the container for a minimum period of 6 hours or over night to detoxify datura alba;
c. taking out and cleaning the Datura alba leaves with demineralized water; and
d. putting the wet dripping leaves through the leaf extractor machine to obtain the aqueous extract.

11. A neem based composition as detoxifying agents (detoxificant) useful for detoxifying toxins that are associated with toxic herbs, used as pharmaceutical, neutraceutical, food and health, wherein the said herbs with high toxicity are pre-treated with neem.

12. A neem based composition as claimed in claim 11, wherein toxic material of the herbs comprises of leaves, stem, root, flower or any other aerial part of the herb.

13. A neem based composition as claimed in claim 11, wherein detoxified herbal extract further formulated into suitable dosage forms.

14. A neem based composition as claimed in claim 11, wherein the herb is Datura alba.
